SEQUENCE LISTING (6 sequences)

SEQ ID NO: 1 - DNA, 21 bp, Artificial Sequence
  Description of Artificial SequencePCR primer
tttcagtatgaattccgcagt

SEQ ID NO: 2 - DNA, 24 bp, Artificial Sequence
  Description of Artificial SequencePCR primer
gcagtaaatcattttgatattaaa

SEQ ID NO: 3 - DNA, 23 bp, Artificial Sequence
  Description of Artificial SequencePCR primer
tgctatgaaaaagatggaaaata

SEQ ID NO: 4 - DNA, 21 bp, Artificial Sequence
  Description of Artificial SequencePCR primer
tgatcctttcctcttttgaat

SEQ ID NO: 5 - DNA, 20 bp, Artificial Sequence
  Description of Artificial SequencePCR primer
caggagaagaacaagaacgc

SEQ ID NO: 6 - DNA, 20 bp, Artificial Sequence
  Description of Artificial SequencePCR primer
cctggctgagggtgtagttg

(19) United States
(12) Patent Application Publication
(10) Pub. No.: US 2003/0079236 A1
(43) Pub. Date: Apr. 24, 2003

(54) Polymorphisms associated with internalizing disorders

(76) Inventors: David E. Comings, Monrovia, CA (US); James P. MacMurray, Loma Linda, CA (US); George Dietz, Pomona, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/304,136

(22) Filed: Nov. 26, 2002

Related U.S. Application Data
(62) Division of application No. 09/657,542, filed on Sep. 8, 2000.
(60) Provisional application No. 60/153,077, filed on Sep. 10, 1999.

(51) Int. Cl. C12Q 1/68; G01N 33/53; G01N 33/567; C07K 16/30

(52) U.S. Cl. 800/3; 435/6; 435/7.2; 530/388.26

(57) ABSTRACT

The present invention is directed to polymorphisms in the MME gene (encoding metalo-membrane endopeptidase, neutral endopeptidase (NEP), enkephalinase) and the ANPEP gene (encoding amino peptidase N (APN), alanyl membrane aminopeptidase) or their gene products and to a process for the diagnosis of internalizing disorders. Internalizing disorders, such as depression, withdrawal, negative affect, anxiety, social problems, phobias, paranoid ideation, alcoholism and interpersonal sensitivity, are diagnosed in accordance with the present invention by analyzing the DNA sequences of the MME and/or ANPEP genes of an individual to be tested and comparing the respective DNA sequence to the known DNA sequence of a normal MME and/or ANPEP gene. Alternatively, the MME and ANPEP genes of an individual to be tested can be screened for mutations which cause internalizing disorders. Prediction of internalizing disorders will enable practitioners to treat internalizing disorders using existing medical therapy.

### CROSS-REFERENCE TO RELATED APPLICATION

The present application is a divisional of U.S. patent application Ser. No. 09/657,542 filed on Sep. 8, 2000. The present application is also related to and claims priority under 35 USC §119(e) to U.S. provisional patent application Serial No. 60/153,077 filed on Sep. 10, 1999. Each application is incorporated herein by reference.

This application was made with Government support under Grant No. RO 1 DA08417 funded by the National Institutes of Health, Bethesda, Md. The federal government may have certain rights in this invention.

### BACKGROUND OF THE INVENTION

The present invention is directed to polymorphisms in the MME gene (encoding metalo-membrane endopeptidase, neutral endopeptidase (NEP), enkephalinase) and the ANPEP gene (encoding amino peptidase N (APN), alanyl membrane aminopeptidase) or their gene products and to a process for the diagnosis of internalizing disorders. Internalizing disorders, such as depression, withdrawal, negative affect, anxiety, social problems, phobias, paranoid ideation, alcoholism and interpersonal sensitivity, are diagnosed in accordance with the present invention by analyzing the DNA sequences of the MME and/or ANPEP genes of an individual to be tested and comparing the respective DNA sequence to the known DNA sequence of a normal MME and/or ANPEP gene. Alternatively, the MME and ANPEP genes of an individual to be tested can be screened for mutations which cause internalizing disorders. Prediction of internalizing disorders will enable practitioners to treat internalizing disorders using existing medical therapy.

The publications and other materials used herein to illuminate the background of the invention or provide additional details respecting the practice, are incorporated by reference, and for convenience are respectively grouped in the appended Lists of References.

Internalizing disorders, such as anxiety, phobias, depression, dysthymia and chronic dissatisfaction with life, constitutes some of the most common and distressing of the psychiatric disorders. The discovery of the endogenous opioids and the genes for their precursors and receptors led to great expectations for the development of new drugs for controlling pain and psychiatric disorders. However, despite a vast literature, compared to dopaminergic, adrenergic and serotonergic systems, the pharmacological manipulation of the opioid system has played only a minor role in the treatment of psychiatric disorders. Because the opioid system is associated with euphoria and the relief of pain, genetically defective opioid pathways could play an important role in the inverse symptoms of chronic depression, anxiety, negative affect and psychic pain.

Thus, there is a continued need to discover genes involved in the opioid pathways which can be used for diagnosis of internalizing disorders and for guiding drug therapy.

### SUMMARY OF THE INVENTION

The present invention is directed to polymorphisms in the MME gene (encoding metalo-membrane endopeptidase, neutral endopeptidase, enkephalinase) and the ANPEP gene (encoding amino peptidase, alanyl membrane aminopeptidase) or the gene products thereof and to a process for the diagnosis of internalizing disorders.

In one aspect, the present invention is directed to one polymorphism in the MME gene which is strongly associated with internalizing behaviors. The polymorphism is a dinucleotide repeat consisting of 6 alleles representing 21 to 26 GT repeats in the 5′ region of the gene.

In a second aspect, the present invention is directed to one polymorphism in the ANPEP gene which is strongly associated with internalizing behaviors. The polymorphism is a single nucleotide polymorphism (SNP) of A257G (gly86arg).

In a third aspect of the invention, analysis of the MME gene and/or ANPEP gene is provided for diagnosis of subjects with internalizing disorders. The diagnostic method comprises analyzing the DNA sequence of the MME gene and/or ANPEP gene of an individual to be tested and comparing it with the DNA sequence of the native, non-variant genes. In a second embodiment, the MME gene and/or ANPEP gene of an individual to be tested is screened for polymorphisms associated with internalizing disorders. The ability to predict internalizing disorders will enable physicians to treat such disorders with appropriate medical therapies.

In a fourth aspect of the invention, subtypes of depression which respond to currently available drugs which inhibit the activity of NEP are identified by analyzing for the presence of MME and ANPEP gene polymorphisms.

In a fifth aspect of the invention, subtypes of depression which respond well to placebo versus those which respond better to active drugs are identified by analyzing for the presence of MME and ANPEP gene polymorphisms.

In a sixth aspect of the present invention, the polymorphisms in the MME and ANPEP genes are used for drug screening and testing.

### BRIEF DESCRIPTION OF THE FIGURES

FIG. 1 shows the distribution of the frequency of the MME alleles in 153 Caucasian controls. The 3 and 4 alleles are the major alleles.

FIG. 2 shows the distribution of the frequency of the MME alleles in the 169 subjects divided into two halves consisting of these with SCL-90 depression scores in the lower half versus those with SCL-90 depression scores in the upper half of the total range. The 4 alleles are associated with low scores while all other alleles are associated with higher scores.

FIG. 3 show the magnitude of the 10 scores of the SCL-90 inventory by MME genotype were 11=4/4, 12=4/non4 and 22=non4/non4. P values are for linear ANOVA. *=those scores that are significantly lower for 22 subjects compared to 11 subjects by the Tukey test at α=0.05.

FIG. 4 shows the additive effect of the MME and ANPEP genes. The MME gene was scored as 4/4/=0, 4/non4=1, and non4/non4=2. The ANPEP gene was scored as 11 and 12=0, and 22=2. The scores of the two genes were added and those with a score of 4 combined with the 3 group. The p values represent linear ANOVA and the r² values (fraction of the variance) was based on linear regression analysis of the MME+ANPEP gene scores versus the SCL-90 scores.

FIGS. 5A-5C show the number and distribution of the MME genotypes against the P300 amplitude for the coronal (FIG. 5A), parietal (FIG. 5B) and frontal (FIG. 5C) leads.

### DETAILED DESCRIPTION OF THE INVENTION

The present invention is directed to polymorphisms in the MME gene (encoding metalo-membrane endopeptidase, neutral endopeptidase (NEP), enkephalinase) and the ANPEP gene (encoding amino peptidase N (APN), alanyl membrane aminopeptidase) or their gene products and to a process for the diagnosis of internalizing disorders. Internalizing disorders, such as depression, withdrawal, negative affect, anxiety, social problems, phobias, paranoid ideation, alcoholism and interpersonal sensitivity, are predicted in accordance with the present invention by analyzing the DNA sequences of the MME and/or ANPEP genes of an individual to be tested and comparing the respective DNA sequence to the known DNA sequence of a normal MME and/or ANPEP gene. Alternatively, the MME and ANPEP genes of an individual to be tested can be screened for mutations which cause internalizing disorders. Prediction of internalizing disorders will enable practitioners to treat internalizing disorders using existing medical therapy. Genetic testing of these polymorphisms is useful for (a) identifying subtypes of depression that will respond to drugs that inhibit NEP activity, (b) identifying subtypes of depression that respond well to placebos versus those that respond better to active drugs and (c) guiding new drug discovery and testing.

The attached Comings et al. “MME and ANPEP in Depression” manuscript describes the analysis of polymorphisms in the MME and ANPEP genes and association with internalizing disorders using the SCL-90 and NEO-Five Factor Personality standardized tests. The manuscript describes the identification of a dinucleotide repeat polymorphism at the MME gene and its association with internalizing disorders, with the most significant association with depression. This manuscript also describes the identification of a single nucleotide polymorphism at the ANPEP gene and its association with internalizing disorders, particularly phobic anxiety. The manuscript further describes the combined effects of these two polymorphisms with respect to internalizing disorders. The most significant additive effects of the two polymorphisms were seen with anxiety, obsessive-compulsive, interpersonal sensitivity and total (SCL-90) scores.

The attached Comings et al. “MME and P300 Wave” manuscript describes the identification of a dinucleotide repeat polymorphism at the MME gene and its association with P300 wave amplitude, low values of which have been linked to substance abuse.

The present invention provides methods of screening the MME and/or ANPEP genes to identify polymorphisms, particularly polymorphisms strongly associated with internalizing disorders. Such methods may further comprise the step of amplifying a portion of the genes, and may further include a step of providing a set of polynucleotides which are primers for amplification of said portion of the genes. The methods are useful for identifying polymorphisms for use in diagnosis and treatment of internalizing disorders.

The present invention provides the information necessary to physicians to select drugs for use in the treatment of internalizing disorders. With the discovery of the association of mutations in the MEM and ANPEP genes, drugs which are known NEP and/or ANP inhibitors can be selected for the treatment of internalizing disorders.

The present invention also provides a method for screening drug candidates to identify drugs useful for treating internalizing disorders. Drug screening is performed by comparing the activity of native genes and those described herein in the presence and absence of potential drugs.

The present invention further provides methods for genotyping individuals with internalizing disorders. Such methods analyze the MME and ANPEP genes for the polymorphisms described herein. The genotyping can include the identification of subtypes of depression that will respond to drugs that inhibit NEP activity, as well as the identification of subtypes of depression that respond well to placebos versus those subtypes that respond better to active drugs. The latter genotyping is particularly useful for testing potential drugs for effects on internalizing disorders, those due to opioid genes and those not due to opioid genes. The genotyping can also include the identification of subtypes of phobic anxiety that will respond to drugs that inhibit APN activity.

Proof that the MME gene and/or ANPEP gene is involved in causing internalizing disorders is obtained by finding polymorphisms or sequences in DNA extracted from affected kindred members which create abnormal MME and/or ANPEP gene products or abnormal levels of the gene products or which are statistically associated with an internalizing disorder. Such internalizing disorder susceptibility alleles will co-segregate with the disease in large kindreds. They will also be present at a much higher frequency in non-kindred individuals with internalizing disorders than in individuals in the general population.

According to the diagnostic and prognostic method of the present invention, alteration of the wild-type MME gene and/or ANPEP gene is detected. In addition, the method can be performed by detecting the wild-type MME gene and/or ANPEP gene and confirming the lack of a cause of an internalizing disorder as a result of these loci. “Alteration of a wild-type gene” encompasses all forms of mutations including deletions, insertions and point mutations in the coding and noncoding regions, particularly those described herein. Deletions may be of the entire gene or of only a portion of the gene. Point mutations may result in stop codons, frameshift mutations or amino acid substitutions. Somatic mutations are those which occur only in certain tissues and are not inherited in the germline. Germline mutations can be found in any of a body's tissues and are inherited. Point mutational events may occur in regulatory regions, such as in the promoter of the gene, leading to loss or diminution of expression of the mRNA. Point mutations may also abolish proper RNA processing, leading to loss of expression of the MME gene product and/or ANPEP gene product, or to a decrease in mRNA stability or translation efficiency.

Useful diagnostic techniques include, but are not limited to fluorescent in situ hybridization (FISH), direct DNA sequencing, PFGE analysis, Southern blot analysis, single stranded conformation analysis (SSCA), RNase protection assay, allele-specific oligonucleotide (ASO), dot blot analysis and PCR-SSCP, as discussed in detail further below. Also useful are the recently developed techniques of mass spectroscopy (such as MALDI or MALDI-TOF; Fu et al., 1998) and DNA microchip technology for the detection of mutations.

The presence of a susceptibility to an internalizing disorder may be ascertained by testing any tissue of a human for polymorphisms or mutations of the MME gene and/or the ANPEP gene. This can be determined by testing DNA from any tissue of the person's body. Most simply, blood can be drawn and DNA extracted from the cells of the blood. In addition, prenatal diagnosis can be accomplished by testing fetal cells, placental cells or amniotic cells for polymorphism or mutations of the MME gene and/or the ANPEP gene. Alteration of a wild-type MME allele and/or wild-type ANPEP allele, whether, for example, by point mutation or deletion, can be detected by any of the means discussed herein.

There are several methods that can be used to detect DNA sequence variation. Direct DNA sequencing, either manual sequencing or automated fluorescent sequencing can detect sequence variation. Another approach is the single-stranded conformation polymorphism assay (SSCP) (Orita et al., 1989). This method does not detect all sequence changes, especially if the DNA fragment size is greater than 200 bp, but can be optimized to detect most DNA sequence variation. The reduced detection sensitivity is a disadvantage, but the increased throughput possible with SSCP makes it an attractive, viable alternative to direct sequencing for mutation detection on a research basis. The fragments which have shifted mobility on SSCP gels are then sequenced to determine the exact nature of the DNA sequence variation. Other approaches based on the detection of mismatches between the two complementary DNA strands include clamped denaturing gel electrophoresis (CDGE) (Sheffield et al., 1991), heteroduplex analysis (HA) (White et al., 1992) and chemical mismatch cleavage (CMC) (Grompe et al., 1989). None of the methods described above will detect large deletions, duplications or insertions, nor will they detect a regulatory mutation which affects transcription or translation of the protein. Other methods which might detect these classes of mutations such as a protein truncation assay or the asymmetric assay, detect only specific types of mutations and would not detect missense mutations. A review of currently available methods of detecting DNA sequence variation can be found in a recent review by Grompe (1993). Once a mutation is known, an allele-specific detection approach such as allele-specific oligonucleotide (ASO) hybridization can be utilized to rapidly screen large numbers of other samples for that same mutation. Such a technique can utilize probes which are labeled with gold nanoparticles to yield a visual color result (Elghanian et al., 1997).

A rapid preliminary analysis to detect polymorphisms in DNA sequences can be performed by looking at a series of Southern blots of DNA cut with one or more restriction enzymes, preferably with a large number of restriction enzymes. Each blot contains a series of normal individuals and a series of LQT cases. Southern blots displaying hybridizing fragments (differing in length from control DNA when probed with sequences near or including the MME locus) indicate a possible mutation. If restriction enzymes which produce very large restriction fragments are used, then pulsed field gel electrophoresis (PFGE) is employed.

Detection of point mutations may be accomplished by molecular cloning of the MME alleles and sequencing the alleles using techniques well known in the art. Also, the gene or portions of the gene may be amplified, e.g., by PCR or other amplification technique, and the amplified gene or amplified portions of the gene may be sequenced.

There are six well known methods for a more complete, yet still indirect, test for confirming the presence of a susceptibility allele: 1) single-stranded conformation analysis (SSCP) (Orita et al., 1989); 2) denaturing gradient gel electrophoresis (DGGE) (Wartell et al., 1990; Sheffield et al., 1989); 3) RNase protection assays (Finkelstein et al., 1990; Kinszler et al., 1991); 4) allele-specific oligonucleotides (ASOs) (Conner et al., 1983); 5) the use of proteins which recognize nucleotide mismatches, such as the E. coli mutS protein (Modrich, 1991); and 6) allele-specific PCR (Ruano and Kidd, 1989). For allele-specific PCR, primers are used which hybridize at their 3′ ends to a particular MME or ANPEP polymorphism or mutation. If the particular polymorphism or mutation is not present, an amplification product is not observed. Amplification Refractory Mutation System (ARMS) can also be used, as disclosed in European Patent Application Publication No. 0332435 and in Newton et al., 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Such a method is particularly useful for screening relatives of an affected individual for the presence of the mutation found in that individual. Other techniques for detecting insertions and deletions as known in the art can be used.

In the first three methods (SSCP, DGGE and RNase protection assay), a new electrophoretic band appears. SSCP detects a band which migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of samples. An example of a mismatch cleavage technique is the RNase protection method. In the practice of the present invention, the method involves the use of a labeled riboprobe which is complementary to the human wild-type MME gene coding sequence. The riboprobe and either mRNA or DNA isolated from the person are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the mRNA or gene but can be a segment of either. If the riboprobe comprises only a segment of the mRNA or gene, it will be desirable to use a number of these probes to screen the whole mRNA sequence for mismatches.

In similar fashion, DNA probes can be used to detect mismatches, through enzymatic or chemical cleavage. See, e.g., Cotton et al., 1988; Shenk et al., 1975; Novack et al., 1986. Alternatively, mismatches can be detected by shifts in the electrophoretic mobility of mismatched duplexes relative to matched duplexes. See, e.g., Cariello, 1988. With either riboprobes or DNA probes, the cellular mRNA or DNA which might contain a mutation can be amplified using PCR (see below) before hybridization. Changes in DNA of the MME gene or ANPEP gene can also be detected using Southern blot hybridization, especially if the changes are gross rearrangements, such as deletions and insertions.

DNA sequences of the MME gene or ANPEP gene which have been amplified by use of PCR may also be screened using allele-specific probes. These probes are nucleic acid oligomers, each of which contains a region of the gene sequence harboring a known mutation. For example, one oligomer may be about 30 nucleotides in length, corresponding to a portion of the gene sequence. By use of a battery of such allele-specific probes, PCR amplification products can be screened to identify the presence of a previously identified mutation in the gene. Hybridization of allele-specific probes with amplified MME or ANPEP sequences can be performed, for example, on a nylon filter. Hybridization to a particular probe under high stringency hybridization conditions indicates the presence of the same mutation in the tissue as in the allele-specific probe.

The newly developed technique of nucleic acid analysis via microchip technology is also applicable to the present invention. In this technique, literally thousands of distinct oligonucleotide probes are built up in an array on a silicon chip. Nucleic acid to be analyzed is fluorescently labeled and hybridized to the probes on the chip. It is also possible to study nucleic acid-protein interactions using these nucleic acid microchips. Using this technique one can determine the presence of mutations or even sequence the nucleic acid being analyzed or one can measure expression levels of a gene of interest. The method is one of parallel processing of many, even thousands, of probes at once and can tremendously increase the rate of analysis. Several papers have been published which use this technique. Some of these are Hacia et al., 1996; Shoemaker et al., 1996; Chee et al., 1996; Lockhart et al., 1996; DeRisi et al., 1996; Lipshutz et al., 1995. This method has already been used to screen individuals for mutations in the breast cancer gene BRCA1 (Hacia et al., 1996). This new technology has been reviewed in a news article in Chemical and Engineering News (Borman, 1996) and been the subject of an editorial (Editorial, Nature Genetics, 1996). Also see Fodor (1997).

The most definitive test for mutations in a candidate locus is to directly compare genomic MME or ANPEP sequences from patients with those from a control population. Alternatively, one could sequence messenger RNA after amplification, e.g., by PCR, thereby eliminating the necessity of determining the exon structure of the candidate gene.

Mutations falling outside the coding region of MME or ANPEP can be detected by examining the non-coding regions, such as introns and regulatory sequences near or within the genes. An early indication that mutations in non-coding regions are important may come from Northern blot experiments that reveal messenger RNA molecules of abnormal size or abundance in patients as compared to those of control individuals.

Alteration of MME or ANPEP mRNA expression can be detected by any techniques known in the art. These include Northern blot analysis, PCR amplification and RNase protection. Diminished mRNA expression indicates an alteration of the wild-type gene. Alteration of wild-type genes can also be detected by screening for alteration of wild-type protein. For example, monoclonal antibodies immunoreactive with MME or APN can be used to screen a tissue. Lack of cognate antigen would indicate a mutation. Antibodies specific for products of mutant alleles could also be used to detect mutant gene product. Such immunological assays can be done in any convenient formats known in the art. These include Western blots, immunohistochemical assays and ELISA assays. Any means for detecting an altered protein can be used to detect alteration of the wild-type MME gene or ANPEP gene. Functional assays, such as protein binding determinations, can be used. In addition, assays can be used which detect MME or APN biochemical function. Finding a mutant MME or ANPEP gene product indicates alteration of a wild-type MME or ANPEP gene.

A mutant MME gene or ANPEP gene or corresponding gene products can also be detected in other human body samples which contain DNA, such as serum, stool, urine and sputum. The same techniques discussed above for detection of mutant genes or gene products in tissues can be applied to other body samples. By screening such body samples, a simple early diagnosis can be achieved for internalizing disorders.

The primer pairs of the present invention are useful for determination of the nucleotide sequence of a particular MME allele or ANPEP allele using PCR. The pairs of single-stranded DNA primers can be annealed to sequences within or surrounding the gene in order to prime amplifying DNA synthesis of the gene itself. A complete set of these primers allows synthesis of all of the nucleotides of the gene coding sequences, i.e., the exons. The set of primers preferably allows synthesis of both intron and exon sequences. Allele-specific primers can also be used. Such primers anneal only to particular MME or ANPEP polymorphic or mutant alleles, and thus will only amplify a product in the presence of the polymorphic or mutant allele as a template.

In order to facilitate subsequent cloning of amplified sequences, primers may have restriction enzyme site sequences appended to their 5′ ends. Thus, all nucleotides of the primers are derived from the gene sequence or sequences adjacent the gene, except for the few nucleotides necessary to form a restriction enzyme site. Such enzymes and sites are well known in the art. The primers themselves can be synthesized using techniques which are well known in the art. Generally, the primers can be made using oligonucleotide synthesizing machines which are commercially available. Given the sequence of each gene and polymorphisms described herein, design of particular primers is well within the skill of the art. The present invention adds to this by presenting data on the intron/exon boundaries thereby allowing one to design primers to amplify and sequence all of the exonic regions completely.

The nucleic acid probes provided by the present invention are useful for a number of purposes. They can be used in Southern blot hybridization to genomic DNA and in the RNase protection method for detecting point mutations already discussed above. The probes can be used to detect PCR amplification products. They may also be used to detect mismatches with the MME or ANPEP gene or mRNA using other techniques.

The presence of an altered (or a mutant) MME gene and/or ANPEP have been associated with internalizing disorders. In order to detect a MME or ANPEP gene polymorphism or mutation, a biological sample is prepared and analyzed for a difference between the sequence of the allele being analyzed and the sequence of the wild-type allele. Polymorphic or mutant alleles can be initially identified by any of the techniques described above. The polymorphic or mutant alleles are then sequenced to identify the specific polymorphism or mutation of the particular allele. Alternatively, polymorphic or mutant alleles can be initially identified by identifying polymorphic or mutant (altered) proteins, using conventional techniques. The alleles are then sequenced to identify the specific polymorphism or mutation for each allele. The polymorphisms or mutations, especially those statistically associated with an internalizing disorder, are then used for the diagnostic and prognostic methods of the present invention.

Definitions

The present invention employs the following definitions, which are, where appropriate, referenced to MME. However, such definitions also are applicable to ANPEP.

“Amplification of Polynucleotides” utilizes methods such as the polymerase chain reaction (PCR), ligation amplification (or ligase chain reaction, LCR) and amplification methods based on the use of Q-beta replicase. Also useful are strand displacement amplification (SDA), thermophilic SDA, and nucleic acid sequence based amplification (3SR or NASBA). These methods are well known and widely practiced in the art. See, e.g., U.S. Pat. Nos. 4,683,195 and 4,683,202 and Innis et al., 1990 (for PCR); Wu and Wallace, 1989 (for LCR); U.S. Pat. Nos. 5,270,184 and 5,455,166 and Walker et al., 1992 (for SDA); Spargo et al., 1996 (for thermophilic SDA) and U.S. Pat. No. 5,409,818, Fahy et al., 1991 and Compton, 1991 for 3SR and NASBA. Reagents and hardware for conducting PCR are commercially available. Primers useful to amplify sequences from the MME region are preferably complementary to, and hybridize specifically to sequences in the MME region or in regions that flank a target region therein. MME sequences generated by amplification may be sequenced directly. Alternatively, but less desirably, the amplified sequence(s) may be cloned prior to sequence analysis. A method for the direct cloning and sequence analysis of enzymatically amplified genomic segments has been described by Scharf et al., 1986.

“Analyte polynucleotide” and “analyte strand” refer to a single- or double-stranded polynucleotide which is suspected of containing a target sequence, and which may be present in a variety of types of samples, including biological samples.

“Antibodies.” The present invention also provides polyclonal and/or monoclonal antibodies and fragments thereof, and immunologic binding equivalents thereof, which are capable of specifically binding to the MME polypeptide and fragments thereof or to polynucleotide sequences from the MME region. The term “antibody” is used both to refer to a homogeneous molecular entity, or a mixture such as a serum product made up of a plurality of different molecular entities. Polypeptides may be prepared synthetically in a peptide synthesizer and coupled to a carrier molecule (e.g., keyhole limpet hemocyanin) and injected over several months into rabbits. Rabbit sera is tested for immunoreactivity to the MME polypeptide or fragment. Monoclonal antibodies may be made by injecting mice with the protein polypeptides, fusion proteins or fragments thereof Monoclonal antibodies will be screened by ELISA and tested for specific immunoreactivity with MME polypeptide or fragments thereof. See, Harlow and Lane, 1988. These antibodies will be useful in assays as well as pharmaceuticals.

Once a sufficient quantity of desired polypeptide has been obtained, it may be used for various purposes. A typical use is in the production of antibodies specific for binding. These antibodies may be either polyclonal or monoclonal, and may be produced by in vitro or in vivo techniques well known in the art. For production of polyclonal antibodies, an appropriate target immune system, typically mouse or rabbit, is selected. Substantially purified antigen is presented to the immune system in a fashion determined by methods appropriate for the animal and by other parameters well known to immunologists. Typical sites for injection are in footpads, intramuscularly, intraperitoneally, or intradermally. Of course, other species may be substituted for mouse or rabbit. Polyclonal antibodies are then purified using techniques known in the art, adjusted for the desired specificity.

An immunological response is usually assayed with an immunoassay. Normally, such immunoassays involve some purification of a source of antigen, for example, that produced by the same cells and in the same fashion as the antigen. A variety of immunoassay methods are well known in the art. See, e.g., Harlow and Lane, 1988, or Goding, 1986.

Monoclonal antibodies with affinities of 10⁻⁸ M⁻¹ or preferably 10⁻⁹ to 10⁻¹⁰ M⁻¹ or stronger will typically be made by standard procedures as described, e.g., in Harlow and Lane, 1988 or Goding, 1986. Briefly, appropriate animals will be selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone tested for their production of an appropriate antibody specific for the desired region of the antigen.

Other suitable techniques involve in vitro exposure of lymphocytes to the antigenic polypeptides, or alternatively, to selection of libraries of antibodies in phage or similar vectors. See Huse et al., 1989. The polypeptides and antibodies of the present invention may be used with or without modification. Frequently, polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced (see U.S. Pat. No. 4,816,567).

“Binding partner” refers to a molecule capable of binding a ligand molecule with high specificity, as for example, an antigen and an antigen-specific antibody or an enzyme and its inhibitor. In general, the specific binding partners must bind with sufficient affinity to immobilize the analyte copy/complementary strand duplex (in the case of polynucleotide hybridization) under the isolation conditions. Specific binding partners are known in the art and include, for example, biotin and avidin or streptavidin, IgG and protein A, the numerous, known receptor-ligand couples, and complementary polynucleotide strands. In the case of complementary polynucleotide binding partners, the partners are normally at least about 15 bases in length, and may be at least 40 bases in length. It is well recognized by those of skill in the art that lengths shorter than 15 (e.g., 8 bases), between 15 and 40, and greater than 40 bases may also be used. The polynucleotides may be composed of DNA, RNA, or synthetic nucleotide analogs. Further binding partners can be identified using, e.g., the two-hybrid yeast screening assay as described herein.

A “biological sample” refers to a sample of tissue or fluid suspected of containing an analyte polynucleotide or polypeptide from an individual including, but not limited to, e.g., plasma, serum, spinal fluid, lymph fluid, the external sections of the skin, respiratory, intestinal, and genitourinary tracts, tears, saliva, blood cells, tumors, organs, tissue and samples of in vitro cell culture constituents.

“Encode”. A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

“Isolated” or “substantially pure”. An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein which has been removed from its naturally occurring environment, and includes recombinant or cloned DNA isolates and chemically synthesized analogs or analogs biologically synthesized by heterologous systems.

“MME Allele” refers, respectively, to normal alleles of the MME locus as well as alleles of MME carrying variations that are associated with an internalizing disorder.

“MME Locus”, “MME Gene”, “MME Nucleic Acids” or “MME Polynucleotide” each refer to polynucleotides, all of which are in the MME region, respectively, that are likely to be expressed in normal tissue, certain alleles of which are associated with an internalizing disorder. The MME locus is intended to include coding sequences, intervening sequences and regulatory elements controlling transcription and/or translation. The MME locus is intended to include all allelic variations of the DNA sequence.

These terms, when applied to a nucleic acid, refer to a nucleic acid which encodes a human NEP polypeptide, fragment, homolog or variant, including, e.g., protein fusions or deletions. The nucleic acids of the present invention will possess a sequence which is either derived from, or substantially similar to a natural NEP-encoding gene or one having substantial homology with a natural NEP-encoding gene or a portion thereof.

The MME gene or nucleic acid includes normal alleles of the MME gene, respectively, including silent alleles having no effect on the amino acid sequence of the NEP polypeptide as well as alleles leading to amino acid sequence variants of the NEP polypeptide that do not substantially affect its function. These terms also include alleles having one or more mutations which adversely affect the function of the NEP polypeptide. A mutation may be a change in the MME nucleic acid sequence which produces a deleterious change in the amino acid sequence of the NEP polypeptide, resulting in partial or complete loss of NEP function, respectively, or may be a change in the nucleic acid sequence which results in the loss of effective NEP expression or the production of aberrant forms of the NEP polypeptide.

The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally-occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The present invention provides recombinant nucleic acids comprising all or part of the MME region. The recombinant construct may be capable of replicating autonomously in a host cell. Alternatively, the recombinant construct may become integrated into the chromosomal DNA of the host cell. Such a recombinant polynucleotide comprises a polynucleotide of genomic, cDNA, semi-synthetic, or synthetic origin which, by virtue of its origin or manipulation, 1) is not associated with all or a portion of a polynucleotide with which it is associated in nature; 2) is linked to a polynucleotide other than that to which it is linked in nature; or 3) does not occur in nature. Where nucleic acid according to the invention includes RNA, reference to the sequence shown should be construed as reference to the RNA equivalent, with U substituted for T.

Therefore, recombinant nucleic acids comprising sequences otherwise not naturally occurring are provided by this invention. Although the wild-type sequence may be employed, it will often be altered, e.g., by deletion, substitution or insertion. cDNA or genomic libraries of various types may be screened as natural sources of the nucleic acids of the present invention, or such nucleic acids may be provided by amplification of sequences resident in genomic DNA or other natural sources, e.g., by PCR. The choice of cDNA libraries normally corresponds to a tissue source which is abundant in mRNA for the desired proteins. Phage libraries are normally preferred, but other types of libraries may be used. Clones of a library are spread onto plates, transferred to a substrate for screening, denatured and probed for the presence of desired sequences.

The DNA sequences used in this invention will usually comprise at least about five codons (15 nucleotides), more usually at least about 7-15 codons, and most preferably, at least about 35 codons. One or more introns may also be present. This number of nucleotides is usually about the minimal length required for a successful probe that would hybridize specifically with a MME-encoding sequence. In this context, oligomers of as low as 8 nucleotides, more generally 8-17 nucleotides, can be used for probes, especially in connection with chip technology.

Techniques for nucleic acid manipulation are described generally, for example, in Sambrook et al., 1989 or Ausubel et al., 1992. Reagents useful in applying such techniques, such as restriction enzymes and the like, are widely known in the art and commercially available from such vendors as New England BioLabs, Boehringer Mannheim, Amersham, Promega, U.S. Biochemicals, New England Nuclear, and a number of other sources. The recombinant nucleic acid sequences used to produce fusion proteins of the present invention may be derived from natural or synthetic sequences. Many natural gene sequences are obtainable from various cDNA or from genomic libraries using appropriate probes. See, GenBank, National Institutes of Health.

As used herein, a “portion” of the MME locus or region or allele is defined as having a minimal size of at least about eight nucleotides, or preferably about 15 nucleotides, or more preferably at least about 25 nucleotides, and may have a minimal size of at least about 40 nucleotides. This definition includes all sizes in the range of 8-40 nucleotides as well as greater than 40 nucleotides. Thus, this definition includes nucleic acids of 8, 12, 15, 20, 25, 40, 60, 80, 100, 200, 300, 400, 500 nucleotides, or nucleic acids having any number of nucleotides within these ranges of values (e.g., 9, 10, 11, 16, 23, 30, 38, 50, 72, 121, etc., nucleotides), or nucleic acids having more than 500 nucleotides.

“NEP protein” or “NEP polypeptide” refers to a protein or polypeptide encoded by the MME locus, variants or fragments thereof. The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring. Ordinarily, such polypeptides will be at least about 50% homologous to the native NEP sequence, preferably in excess of about 90%, and more preferably at least about 95% homologous. Also included are proteins encoded by DNA which hybridize under high or low stringency conditions, to NEP-encoding nucleic acids and closely related polypeptides or proteins retrieved by antisera to the NEP protein(s).

The NEP polypeptide may be in isolated and/or purified form, free or substantially free of material with which it is naturally associated. The polypeptide may, if produced by expression in a prokaryotic cell or produced synthetically, lack native post-translational processing, such as glycosylation. Alternatively, the present invention is also directed to polypeptides which are sequence variants, alleles or derivatives of the NEP polypeptide. Such polypeptides may have an amino acid sequence which differs from the wild-type by one or more of addition, substitution, deletion or insertion of one or more amino acids.

“Operably linked” refers to a juxtaposition wherein the components so described are in a relationship permitting them to function in their intended manner. For instance, a promoter is operably linked to a coding sequence if the promoter affects its transcription or expression.

The terms “peptide mimetic” or “mimetic” are intended to refer to a substance which has the essential biological activity of the NEP polypeptide. A peptide mimetic may be a peptide-containing molecule that mimics elements of protein secondary structure (Johnson et al., 1993). The underlying rationale behind the use of peptide mimetics is that the peptide backbone of proteins exists chiefly to orient amino acid side chains in such a way as to facilitate molecular interactions, such as those of antibody and antigen, enzyme and substrate or scaffolding proteins. A peptide mimetic is designed to permit molecular interactions similar to the natural molecule. A mimetic may not be a peptide at all, but it will retain the essential biological activity of natural NEP polypeptide.

“Probes”. Polynucleotide polymorphisms associated with MME alleles which are associated with internalizing disorders are detected by hybridization with a polynucleotide probe which forms a stable hybrid with that of the target sequence, under stringent to moderately stringent hybridization and wash conditions. If it is expected that the probes will be perfectly complementary to the target sequence, high stringency conditions will be used. Hybridization stringency may be lessened if some mismatching is expected, for example, if variants are expected with the result that the probe will not be completely complementary. Conditions are chosen which rule out non-specific/adventitious bindings, that is, which minimize noise. (It should be noted that, throughout this disclosure, if it is stated simply that “stringent” conditions are used, that it is meant to be read that “high stringency” conditions are used.) Since such indications identify neutral DNA polymorphisms as well as mutations, these indications need further analysis to demonstrate detection of a MME susceptibility allele.

Probes for MME alleles may be derived from the sequences of the MME region, its cDNA, functionally equivalent sequences, or the complements thereof. The probes may be of any suitable length, which span all or a portion of the MME region, and which allow specific hybridization to the region. If the target sequence contains a sequence identical to that of the probe, the probes may be short, e.g., in the range of about 8-30 base pairs, since the hybrid will be relatively stable under even stringent conditions. If some degree of mismatch is expected with the probe, i.e., if it is suspected that the probe will hybridize to a variant region, a longer probe may be employed which hybridizes to the target sequence with the requisite specificity.

The probes will include an isolated polynucleotide attached to a label or reporter molecule and may be used to isolate other polynucleotide sequences, having sequence similarity by standard methods. For techniques for preparing and labeling probes see, e.g., Sambrook et al., 1989 or Ausubel et al., 1992. Other similar polynucleotides may be selected by using homologous polynucleotides. Alternatively, polynucleotides encoding these or similar polypeptides may be synthesized or selected by use of the redundancy in the genetic code. Various codon substitutions maybe introduced, e.g., by silent changes (thereby producing various restriction sites) or to optimize expression for a particular system. Mutations may be introduced to modify the properties of the polypeptide, perhaps to change the polypeptide degradation or turnover rate.

Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

Portions of the polynucleotide sequence having at least about eight nucleotides, usually at least about 15 nucleotides, and fewer than about 6 kb, usually fewer than about 1.0 kb, from a polynucleotide sequence encoding MME are preferred as probes. This definition therefore includes probes of sizes 8 nucleotides through 6000 nucleotides. Thus, this definition includes probes of 8, 12, 15, 20, 25, 40, 60, 80, 100, 200, 300, 400 or 500 nucleotides or probes having any number of nucleotides within these ranges of values (e.g., 9, 10, 11, 16, 23, 30, 38, 50, 72, 121, etc., nucleotides), or probes having more than 500 nucleotides. The probes may also be used to determine whether mRNA encoding MME is present in a cell or tissue. The present invention includes all novel probes having at least 8 nucleotides, its complement or functionally equivalent nucleic acid sequences. The present invention does not include probes which exist in the prior art.

Similar considerations and nucleotide lengths are also applicable to primers which may be used for the amplification of all or part of the MME gene. Thus, a definition for primers includes primers of 8, 12, 15, 20, 25, 40, 60, 80, 100, 200, 300, 400, 500 nucleotides, or primers having any number of nucleotides within these ranges of values (e.g., 9, 10, 11, 16, 23, 30, 38, 50, 72, 121, etc. nucleotides), or primers having more than 500 nucleotides, or any number of nucleotides between 500 and 6000. The primers may also be used to determine whether mRNA encoding MME is present in a cell or tissue. The present invention includes all novel primers having at least 8 nucleotides derived from the MME locus for amplifying the MME gene, its complement or functionally equivalent nucleic acid sequences. The present invention does not include primers which exist in the prior art. That is, the present invention includes all primers having at least 8 nucleotides with the proviso that it does not include primers existing in the prior art.

“Protein modifications or fragments” are provided by the present invention for NEP polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as ³²P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods of labeling polypeptides are well known in the art. See Sambrook et al., 1989 or Ausubel et al., 1992.

Besides substantially full-length polypeptides, the present invention provides for biologically active fragments of the polypeptides. Significant biological activities include ligand-binding, immunological activity and other biological activities characteristic of NEP polypeptides. Immunological activities include both immunogenic function in a target immune system, as well as sharing of immunological epitopes for binding, serving as either a competitor or substitute antigen for an epitope of the NEP protein. As used herein, “epitope” refers to an antigenic determinant of a polypeptide. An epitope could comprise three amino acids in a spatial conformation which is unique to the epitope. Generally, an epitope consists of at least five such amino acids, and more usually consists of at least 8-10 such amino acids. Methods of determining the spatial conformation of such amino acids are known in the art.

For immunological purposes, tandem-repeat polypeptide segments may be used as immunogens, thereby producing highly antigenic proteins. Alternatively, such polypeptides will serve as highly efficient competitors for specific binding. Production of antibodies specific for NEP polypeptides or fragments thereof is described below.

The present invention also provides for fusion polypeptides, comprising NEP polypeptides and fragments. Homologous polypeptides may be fusions between two or more NEP polypeptide sequences or between the sequences of NEP and a related protein. Likewise, heterologous fusions may be constructed which would exhibit a combination of properties or activities of the derivative proteins. For example, ligand-binding or other domains may be “swapped” between different new fusion polypeptides or fragments. Such homologous or heterologous fusion polypeptides may display, for example, altered strength or specificity of binding. Fusion partners include immunoglobulins, bacterial β-galactosidase, trpE, protein A, β-lactamase, alpha amylase, alcohol dehydrogenase and yeast alpha mating factor. See Godowski et al., 1988.

Fusion proteins will typically be made by either recombinant nucleic acid methods, as described below, or may be chemically synthesized. Techniques for the synthesis of polypeptides are described, for example, in Merrifield (1963).

“Protein purification” refers to various methods for the isolation of the NEP polypeptides from other biological material, such as from cells transformed with recombinant nucleic acids encoding NEP, and are well known in the art. For example, such polypeptides may be purified by immunoaffinity chromatography employing, e.g., the antibodies provided by the present invention. Various methods of protein purification are well known in the art, and include those described in Deutscher, 1990 and Scopes, 1982.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

A NEP protein is substantially free of naturally associated components when it is separated from the native contaminants which accompany it in its natural state. Thus, a polypeptide which is chemically synthesized or synthesized in a cellular system different from the cell from which it naturally originates will be substantially free from its naturally associated components. A protein may also be rendered substantially free of naturally associated components by isolation, using protein purification techniques well known in the art.

A polypeptide produced as an expression product of an isolated and manipulated genetic sequence is an “isolated polypeptide”, as used herein, even if expressed in a homologous cell type. Synthetically made forms or molecules expressed by heterologous cells are inherently isolated molecules.

“Recombinant nucleic acid” is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

“Regulatory sequences” refers to those sequences normally within 100 kb of the coding region of a locus, but they may also be more distant from the coding region, which affect the expression of the gene (including transcription of the gene, and translation, splicing, stability or the like of the messenger RNA).

“Substantial homology or similarity”. A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

To determine homology between two different nucleic acids, the percent homology is to be determined using the BLASTN program “BLAST 2 sequences”. This program is available for public use from the National Center for Biotechnology Information (NCBI) over the Internet (http://www.ncbi.nlm.nih.gov/gorf/b12.html) (Altschul et al., 1997). The parameters to be used are whatever combination of the following yields the highest calculated percent homology, with the default parameters shown in parentheses:

Program—blastn

Matrix—0 BLOSUM62

Reward for a match—0 or 1 (1)

Penalty for a mismatch—0, −1, −2 or −3 (−2)

Open gap penalty—0, 1, 2, 3, 4 or 5 (5)

Extension gap penalty—0 or 1 (1)

Gap x_dropoff—0 or 50 (50)

Expect—10

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. See, Kanehisa, 1984. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Wetmur and Davidson, 1968.

Probe sequences may also hybridize specifically to duplex DNA under certain conditions to form triplex or other higher order DNA complexes. The preparation of such probes and suitable hybridization conditions are well known in the art.

The terms “substantial homology” or “substantial identity”, when referring to polypeptides, indicate that the polypeptide or protein in question exhibits at least about 30% identity with an entire naturally-occurring protein or a portion thereof, usually at least about 70% identity, more usually at least about 80% identity, preferably at least about 90% identity, and more preferably at least about 95% identity.

Homology, for polypeptides, is typically measured using sequence analysis software. See, e.g., the Sequence Analysis Software Package of the Genetics Computer Group, University of Wisconsin Biotechnology Center, 910 University Avenue, Madison, Wis. 53705. Protein analysis software matches similar sequences using measures of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

“Substantially similar function” refers to the function of a modified nucleic acid or a modified protein, with reference to the wild-type MME nucleic acid or wild-type NEP polypeptide. The modified polypeptide will be substantially homologous to the wild-type NEP polypeptide and will have substantially the same function. The modified polypeptide may have an altered amino acid sequence and/or may contain modified amino acids. In addition to the similarity of function, the modified polypeptide may have other useful properties, such as a longer half-life. The similarity of function (activity) of the modified polypeptide may be substantially the same as the activity of the wild-type NEP polypeptide. Alternatively, the similarity of function (activity) of the modified polypeptide may be higher than the activity of the wild-type NEP polypeptide. The modified polypeptide is synthesized using conventional techniques, or is encoded by a modified nucleic acid and produced using conventional techniques. The modified nucleic acid is prepared by conventional techniques. A nucleic acid with a function substantially similar to the wild-type MME gene function produces the modified protein described above.

A polypeptide “fragment”, “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids.

The polypeptides of the present invention, if soluble, may be coupled to a solid-phase support, e.g., nitrocellulose, nylon, column packing materials (e.g., Sepharose beads), magnetic beads, glass wool, plastic, metal, polymer gels, cells, or other substrates. Such supports may take the form, for example, of beads, wells, dipsticks, or membranes.

“Target region” refers to a region of the nucleic acid which is amplified and/or detected. The term “target sequence” refers to a sequence with which a probe or primer will form a stable hybrid under desired conditions.

The practice of the present invention employs, unless otherwise indicated, conventional techniques of chemistry, molecular biology, microbiology, recombinant DNA, genetics, and immunology. See, e.g., Maniatis et al., 1982; Sambrook et al., 1989; Ausubel et al., 1992; Glover, 1985; Anand, 1992; Guthrie and Fink, 1991. A general discussion of techniques and materials for human gene mapping, including mapping of human chromosome 1, is provided, e.g., in White and Lalouel, 1988.

Recombinant or chemically synthesized nucleic acids or vectors, transformation or transfection of host cells, transformed or transfected host cells and polypeptides are produced using conventional techniques, such as described in U.S. Pat. Nos. 5,837,492; 5,800,998 and 5,891,628, each incorporated herein by reference.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art, including those described in U.S. Pat. Nos. 5,837,492; 5,800,998 and 5,891,628, each incorporated herein by reference.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing are generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Briefly, a method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Prior to, or as well as being screened for modulation of activity, test substances may be screened for ability to interact with the polypeptide, e.g., in a yeast two-hybrid system (e.g., Bartel et al., 1993; Fields and Song, 1989; Chevray and Nathans, 1992; Lee et al., 1995). This system may be used as a coarse screen prior to testing a substance for actual ability to modulate activity of the polypeptide. Alternatively, the screen could be used to screen test substances for binding to an NEP or APN specific binding partner, or to find mimetics of the NEP or APN polypeptide.

Following identification of a substance which modulates or affects polypeptide activity, the substance may be further investigated. Furthermore, it may be manufactured and/or used in preparation, i.e., a manufacture or formulation, or a composition such as a medicament, pharmaceutical composition or drug. These may be administered to individuals.

In order to detect the presence of an MME or ANPEP allele predisposing an individual to an internalizing disorder, a biological sample such as blood is prepared and analyzed for the presence or absence of susceptibility alleles of MME or ANPEP. In order to detect the presence of an internalizing disorder or as a prognostic indicator, a biological sample is prepared and analyzed for the presence or absence of polymorphic or mutant alleles of MME or ANPEP. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis. Suitable diagnostic techniques include those described herein as well as those described in U.S. Pat. Nos. 5,837,492; 5,800,998 and 5,891,628, each incorporated herein by reference.

Initially, the screening method involves amplification of the relevant MME or ANPEP sequences. In another preferred embodiment of the invention, the screening method involves a non-PCR based strategy. Such screening methods include two-step label amplification methodologies that are well known in the art. Both PCR and non-PCR based screening strategies can detect target sequences with a high level of sensitivity.

The most popular method used today is target amplification. Here, the target nucleic acid sequence is amplified with polymerases. One particularly preferred method using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be sequenced or used as a substrate for DNA probes.

When the probes are used to detect the presence of the target sequences the biological sample to be analyzed, such as blood or serum, may be treated, if desired, to extract the nucleic acids. The sample nucleic acid may be prepared in various ways to facilitate detection of the target sequence, e.g. denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques known in the art.

Analyte nucleic acid and probe are incubated under conditions which promote stable hybrid formation of the target sequence in the probe with the putative targeted sequence in the analyte. The region of the probes which is used to bind to the analyte can be made completely complementary to the targeted region of MME or ANPEP. Therefore, high stringency conditions are desirable in order to prevent false positives. However, conditions of high stringency are used only if the probes are complementary to regions of the chromosome which are unique in the genome. The stringency of hybridization is determined by a number of factors during hybridization and during the washing procedure, including temperature, ionic strength, base composition, probe length, and concentration of formamide. These factors are outlined in, for example, Maniatis et al., 1982 and Sambrook et al., 1989. Under certain circumstances, the formation of higher order hybrids, such as triplexes, quadraplexes, etc., may be desired to provide the means of detecting target sequences.

Detection of the resulting hybrid, if any, is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies, gold nanoparticles and the like. Variations of this basic scheme are known in the art, and include those variations that facilitate separation of the hybrids to be detected from extraneous materials and/or that amplify the signal from the labeled moiety. A number of these variations are reviewed in, e.g., Matthews and Kricka, 1988; Landegren et al., 1988; Mifflin, 1989; U.S. Pat. No. 4,868,105; and in EPO Publication No. 225,807.

As noted above, non-PCR based screening assays are also contemplated in this invention. This procedure hybridizes a nucleic acid probe (or an analog such as a methyl phosphonate backbone replacing the normal phosphodiester), to the low level DNA target. This probe may have an enzyme covalently linked to the probe, such that the covalent linkage does not interfere with the specificity of the hybridization. This enzyme-probe-conjugate-target nucleic acid complex can then be isolated away from the free probe enzyme conjugate and a substrate is added for enzyme detection. Enzymatic activity is observed as a change in color development or luminescent output resulting in a 10³-10⁶ increase in sensitivity. For an example relating to the preparation of oligodeoxynucleotide-alkaline phosphatase conjugates and their use as hybridization probes, see Jablonski et al. (1986).

Two-step label amplification methodologies are known in the art. These assays work on the principle that a small ligand (such as digoxigenin, biotin, or the like) is attached to a nucleic acid probe capable of specifically binding MME or ANPEP. Allele-specific probes are also contemplated within the scope of this example, and exemplary allele-specific probes include probes encompassing the predisposing mutations of this patent application.

In one example, the small ligand attached to the nucleic acid probe is specifically recognized by an antibody-enzyme conjugate. In one embodiment of this example, digoxigenin is attached to the nucleic acid probe. Hybridization is detected by an antibody-alkaline phosphatase conjugate which turns over a chemiluminescent substrate. For methods for labeling nucleic acid probes according to this embodiment see Martin et al., 1990. In a second example, the small ligand is recognized by a second ligand-enzyme conjugate that is capable of specifically complexing to the first ligand. A well known embodiment of this example is the biotin-avidin type of interactions. For methods for labeling nucleic acid probes and their use in biotin-avidin based assays see Rigby et al., 1977 and Nguyen et al., 1992.

The presence of an internalizing disorder can also be detected on the basis of the alteration of wild-type NEP or APN polypeptide. Such alterations can be determined by sequence analysis in accordance with conventional techniques. More preferably, antibodies (polyclonal or monoclonal) are used to detect differences in, or the absence of NEP or APN peptides. Techniques for raising and purifying antibodies are well known in the art, and any such techniques may be chosen to achieve the preparations claimed in this invention. In a preferred embodiment of the invention, antibodies will immunoprecipitate NEP or APN proteins from solution as well as react with these proteins on Western or immunoblots of polyacrylamide gels. In another preferred embodiment, antibodies will detect NEP or APN proteins in paraffin or frozen tissue sections, using immunocytochemical techniques.

Preferred embodiments relating to methods for detecting NEP or APN or its polymorphisms/mutations include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference.

According to the present invention, a method is also provided of supplying wild-type MME function and/or wild-type ANPEP function to a cell which carries a mutant MME allele, respectively. Supplying such a function should allow normal functioning of the recipient cells. The wild-type gene or a part of the gene may be introduced into the cell in a vector such that the gene remains extrachromosomal. In such a situation, the gene will be expressed by the cell from the extrachromosomal location. More preferred is the situation where the wild-type gene or a part thereof is introduced into the mutant cell in such a way that it recombines with the endogenous mutant gene present in the cell. Such recombination requires a double recombination event which results in the correction of the gene mutation. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the practitioner. Conventional methods are employed, including those described in U.S. Pat. Nos. 5,837,492; 5,800,998 and 5,891,628, each incorporated herein by reference.

Alternatively, peptides which have NEP activity and/or APN activity can be supplied to cells which carry a mutant or missing MME allele and/or ANPEP allele. Protein can be produced by expression of the cDNA sequence in bacteria, for example, using known expression vectors. Alternatively, the polypeptide(s) can be extracted from polypeptide-producing mammalian cells. In addition, the techniques of synthetic chemistry can be employed to synthesize the protein. Any of such techniques can provide the preparation of the present invention which comprises the NEP protein and/or APN protein. The preparation is substantially free of other human proteins. This is most readily accomplished by synthesis in a microorganism or in vitro. Active NEP and/or APN molecules can be introduced into cells by microinjection or by use of liposomes, for example. Alternatively, some active molecules may be taken up by cells, actively or by diffusion. Conventional methods are employed, including those described in U.S. Pat. Nos. 5,837,492; 5,800,998 and 5,891,628, each incorporated herein by reference.

Animals for testing therapeutic agents or for developing animal and cellular models can be selected after mutagenesis of whole animals or after treatment of germline cells or zygotes. Such treatments include insertion of polymorphic/mutant MME alleles and/or ANPEP alleles, usually from a second animal species, as well as insertion of disrupted homologous genes. Alternatively, the endogenous MME gene and/or ANPEP gene of the animals may be disrupted by insertion or deletion mutation or other genetic alterations using conventional techniques (Capecchi, 1989; Valancius and Smithies, 1991; Hasty et al., 1991; Shinkai et al., 1992; Mombaerts et al., 1992; Philpott et al., 1992; Snouwaert et al., 1992; Donehower et al., 1992). These transgenic, transplacement and knock-out animals can also be used to screen drugs that may influence the biochemical, neuropathological, and behavioral parameters relevant to internalizing disorders. Cell lines can also be derived from these animals for use as cellular models, or in drug screening. Conventional methods are employed, including those described in U.S. Pat. Nos. 5,837,492; 5,800,998 and. 5,891,628, each incorporated herein by reference.

The identification of the association between the MME gene polymorphism/mutations and internalizing disorders and/or the association between the ANPEP gene polymorphism/mutations and internalizing disorders permits the early presymptomatic screening of individuals to identify those at risk for developing internalizing disorders or to identify the cause of such disorders. To identify such individuals, the alleles are screened as described herein or using conventional techniques, including but not limited to, one of the following methods: fluorescent in situ hybridization (FISH), direct DNA sequencing, PFGE analysis, Southern blot analysis, single stranded conformation analysis (SSCP), linkage analysis, RNase protection assay, allele-specific oligonucleotide (ASO), dot blot analysis and PCR-SSCP analysis. Also useful is the recently developed technique of DNA microchip technology. Such techniques are described in U.S. Pat. Nos. 5,837,492, 5,800,998 and 5,891,628, each incorporated herein by reference.

Genetic testing will enable practitioners to identify individuals at risk for internalizing disorders at, or even before, birth. Presymptomatic diagnosis will enable better treatment of these disorders, including the use of existing medical therapies. Genetic testing will also enable practitioners to identify individuals having diagnosed internalizing disorders those in which the diagnosis results from MME and/or ANPEP polymorphisms. Genotyping of such individuals will be useful for (a) identifying subtypes of depression that will respond to drugs that inhibit NEP activity, (b) identifying subtypes of depression that respond well to placebos versus those that respond better to active drugs and (c) guide new drug discovery and testing. This genotyping is particularly useful, since 30% to 50% of antidepressant drug response results from a placebo response which may be caused by the present genes.

The NEP and/or APN polypeptides, antibodies, peptides and nucleic acids of the present invention can be formulated in pharmaceutical compositions, which are prepared according to conventional pharmaceutical compounding techniques. See, for example, Remington 's Pharmaceutical Sciences, 18th Ed. (1990, Mack Publishing Co., Easton, Pa.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g., intravenous, oral, intrathecal, epineural or parenteral.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington 's Pharmaceutical Sciences.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

Instead of administering these agents directly, they could be produced in the target cell, e.g. in a viral vector such as described above or in a cell based delivery system such as described in U.S. Pat. No. 5,550,050 and published PCT application Nos. WO 92/19195, WO 94/25503, WO 95/01203, WO 95/05452, WO 96/02286, WO 96/02646, WO 96/40871, WO 96/40959 and WO 97/12635, designed for implantation in a patient. The vector could be targeted to the specific cells to be treated, or it could contain regulatory elements which are more tissue specific to the target cells. The cell based delivery system is designed to be-implanted in a patient's body at the desired target site and contains a coding sequence for the active agent. Alternatively, the agent could be administered in a precursor form for conversion to the active form by an activating agent produced in, or targeted to, the cells to be treated. See for example, EP 425,731A and WO 90/07936.

### EXAMPLES

The present invention is further described with reference to the following examples, which are offered by way of illustration and is not intended to limit the invention in any manner. Standard techniques well known in the art or the techniques specifically described therein were utilized.

### Example 1

### Methods

Subjects. The test subjects consisted of two groups, student controls and subjects on an addiction treatment unit (ATU).

Student Sample. The student sample consisted of 153 non-Hispanic Caucasian students recruited from a Southern California university. There were 48 percent males (n=74) and 52 percent females (n=79). The ages ranged from 23 to 49 years with a mean of 33.4, S.D.=7.9. The older mean age of these students was due to their having been recruited as an age-matched sample for comparison with the ATU sample.

ATU Sample. The Addiction Treatment Unit is the inpatient addiction treatment center of the Jerry L. Pettis Veterans Administration Hospital in Loma Linda, Calif. Between 1994 and 1997, all new admissions to the ATU that give informed consent were entered into a National Institute of Drug Abuse sponsored study of genetic factors in drug abuse/dependence. The present study included 95 male non-Hispanic Caucasian ATU subjects. The mean age of the ATU subjects was 41.0 years, S.D. 7.3.

In both the student and ATU groups, after obtaining written informed consent, a blood sample was obtained for genetic studies and subjects were administered a number of standardized tests including the SCL-90 (Steer et al., 1994; Kass et al., 1983) and NEO-Five Factor Personality Inventory (NEO-FFI) (Costa & McCrae, 1992). Since the SCL-90 is highly dependent upon subject's personality features and less correlated with clinicians ratings (Kass et al., 1983), the present results should be interpreted bearing this in mind. To minimize the effects of gender due to the presence of females in the student but not the ATU sample, we present the results on males only (n=169).

MME polymorphism. The sequence of the MME (CD10, neutral endopeptidase 24.11) gene shows a GT repeat in the 5′ region of the gene (Haouas et al., 1995). PCR amplification showed the presence of a polymorphism of this region consisting of 6 MME representing 21 to 26 GT repeats. The use of three forward primers:

TTTCAGTATGAATTCCGCAGT (SEQ ID NO:1),

GCAGTAAATCATTTTGATATTAAA (SEQ ID NO:2), and

TGCTATGAAAAAGATGGAAAATA (SEQ ID NO:3),

and a single fluorescent labeled (HEX Amidite, Applied Biosystems, Foster City, Calif.) reverse primer:

TGATCCTTTCCTCTTTTGAAT SEQ ID NO:4),

allowed the analysis of three samples on a single well of the Applied Biosystems 373 DNA sequencer.

The PCR reaction was performed under the solution conditions described for the Qiagen PCR Kit (Valencia, Calif.), but not including the Q solution. To a final volume of 14 ul of reaction mixture was added 50 ng of human DNA. The thermocycling protocol consisted of an initial denaturation at 95 degrees C. for 5 minutes; a cycle of 95 degrees for 30 seconds, then one minute at 55 degrees C. and one minute at 72 degrees C. repeated 38 times, ending with an incubation at 72 degrees C. for 5 minutes. Two μl of the 10 fold diluted PCR product was then added to 2.5 μl deionized formamide and 0.5 μl of ROX 500 standard (Applied Biosystems, Foster City, Calif.), denatured for 2 min at 92° C. and loaded on 6% denaturing polyacrylamide gel. The gel was electrophoresed for 5 hours at a constant 25 W. The gel was laser scanned and analyzed using the internal ROX 500 standards present in each lane. The peaks were recognized by Genotyper (version 1.1) based on the color fragments sized by base pair length.

ANPEP polymorphism. Based on the sequencing of the human aminopeptidase (EC 3.4.11.2) gene (Watt & Willard, 1990), we identified a 257 G (gly 86 arg) polymorphism. The PCR primers were

forward: CAGGAGAAGAACAAGAACGC (SEQ ID NO:5), and

reverse: CCTGGCTGAGGGTGTAGTTG (SEQ ID NO:6).

The PCR conditions were the same as for the MME polymorphism. The thermocycling protocol consisted of an initial denaturation at 95 degrees C. for 5 minutes; a cycle of 95 degrees for 30 seconds, one minute at 55 degrees C., and one minute at 72 degrees C., repeated 38 times, and ending with an incubation at 72 degrees C. for 5 minutes. The PCR reaction was mixed with 10 micro L of 1× buffer 2(New England Biolabs, Beverly, Mass.) which had been supplemented to 20 mM MgCl₂, and included 2 u of Msp I enzyme and incubated over night at 37 degrees C. These PCR conditions produced a 300 bp product with the polymorphism in the center. When cut with Msp I both products were 150 bp resulting in an enhanced signal. The products were electrophoresed in 2% agarose.

Statistics. ANOVA was used to assess the potential association between the MME and ANPEP genes and the SCL-90 total score and subscores, and the negative affect subscore of the NEO-FFI (Saucier, 1998). The MME alleles were placed in three groups, 4/4, non4/non4, and non4/non4 (see below). The ANPEP alleles were placed in three groups, gly/gly, gly/arg, arg/arg. To examine the additive effect of the two genes, the three genotypes of each gene were scored 0, 1 or 2 based on the ANOVA results, and for each individual the two scores were added. This initially formed a score from 0 to 4. However, since the number of cases in with a score of 3 and 4 was small (10 and 13 respectively) these were merged to from a combined score of 0 to 3 and the magnitude of the SCL-90 scores analyzed by ANOVA. In each case the p values were based on ANOVA for a linear trend. A Tukey test was included in the analysis to identify groups that were significantly different at α=0.05. Linear regression analysis was performed to determine the percent of the variance (r²) due to the combined genes. The above statistical analyses utilized the SPSS Statistical Package (SPSS, Inc, Chicago, Ill.). The frequency of the MME alleles in subjects with SCL-90 scores in the lower half of the total versus those in the upper half were examined by exact chi square analysis using StatXact Software (Cytel Software Corporation, Cambridge, Mass.). The <3 alleles were combined with the 3 allele group and the >5 alleles were combined with the 5 allele group.

### Example 2

### MME Allele Frequencies

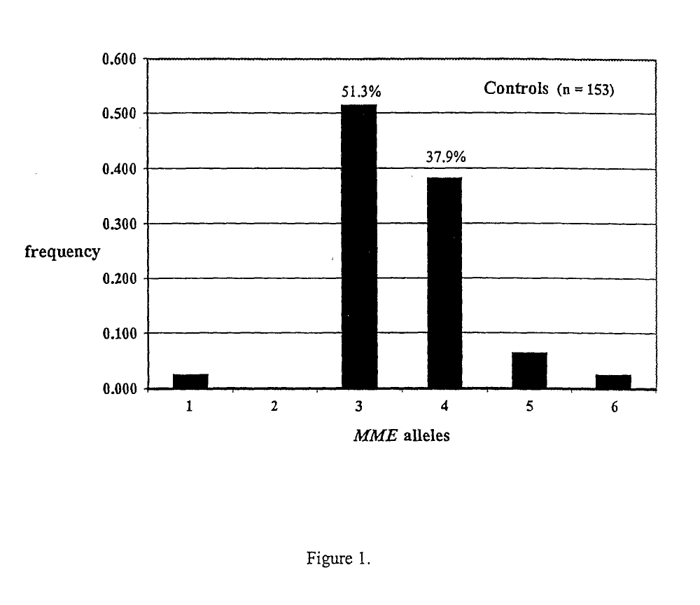

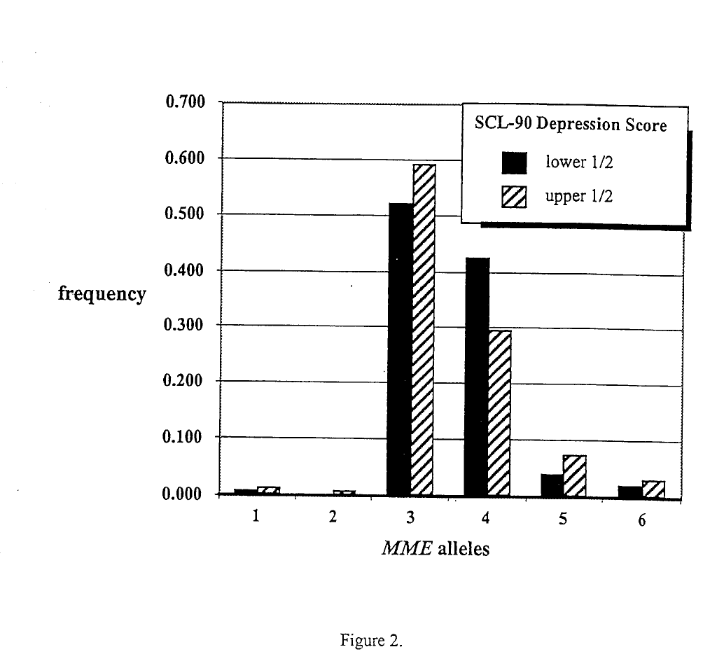

The distribution of the frequencies of these alleles in the 153 controls (both sexes) in the student sample is shown in FIG. 1. The two major alleles, 3 and 4 represented 51.3 and 37.9% of the total respectively. Together they accounted for the 89.2% of the alleles. FIG. 2 shows the distribution of the alleles in the test sample of 169 males consisting of 74 student controls and 95 ATU subjects. The frequency of the 4 allele was increased in those whose SCL-90 depression scores were in the lower half of all the scores. All of the remaining alleles were increased in frequency in those subjects whose SCL-90 depression scores were in the upper half of the total scores. These were significant with exact chi square=7.31, d.f.=2, p=0.026. Since the same pattern was present for the SCL-90 depression score in a totally independent set of subjects consisting of the 79 Caucasian female students (see above) plus an additional 10 female students of other races, for further analysis we grouped the MME genotypes into three groups consisting of 4/4, 4/non4 and non4/non4.

### Example 3

### MME Genotypes and SCL-90 Scores

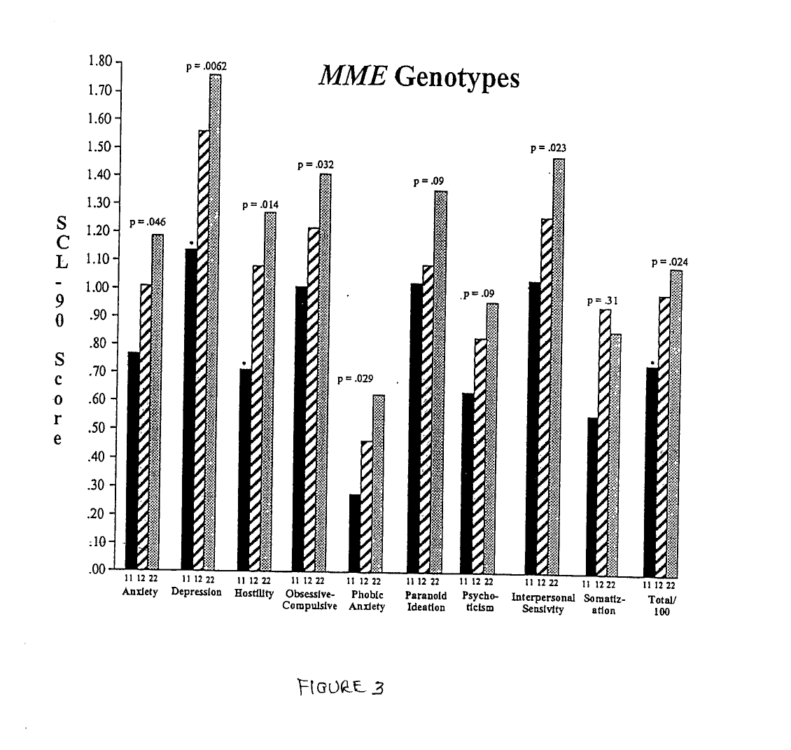

Based on the results of Huss et al. (1998), we performed an initial, exploratory MANOVA using the SCL-90 depression and anxiety subscores, and the negative affect subscore of the NEO-FFI against the total set of controls and ATU subjects. Age, sex, and diagnosis (control versus ATU) were used as covariates. This was significant at p<0.05. We then progressed to ANOVA of each of the SCL-90 subscores against the MME genotypes. FIG. 3 shows the distribution of all of the SCL-90 scores for the total set of 169 subjects by MME genotype, where 11=4/4, 12=4/non4, and 22=non4/non4. There was a trend for a linear increase in the scores across the three genotype groups from 4/4 to non4/non4. The linear trend p values were significant for anxiety (0.046), depression (0.0062), hostility (0.014), obsessive-compulsive (0.032), phobic anxiety (0.029), interpersonal sensitivity (0.023) and the total score (0.024). The gene scores for the MME genotypes were 4/4=0, 4/non4=1 and non4/non4=2. No Bonferroni correction was made for the number of variables examined. However, since 7 of the 10 scores were significant at p<0.05, it was unlikely the results were due to chance.

### Example 4

### ANPEP Genotypes and SCL-90 Scores

The 11 genotype=gly/gly, the 12=gly/arg, and 22=arg/arg. The results for standard or linear ANOVA were not significant for any of the scores except phobic anxiety (p=0.048). However, for all scores except somatization, the values with highest for those carrying the 22 genotype, and with the exception of the hostility score, the values for those with the 22 genotype were the highest while those for the 11 and 22 genotypes were similar. Thus, the ANPEP gene was scored as 11=0, 12=0, and 22=2. The p values were less than 0.20 for anxiety, obsessive compulsive, phobic anxiety, and interpersonal sensitivity.

### Example 5

### Additive Effect of the MME and ANPEP Genes

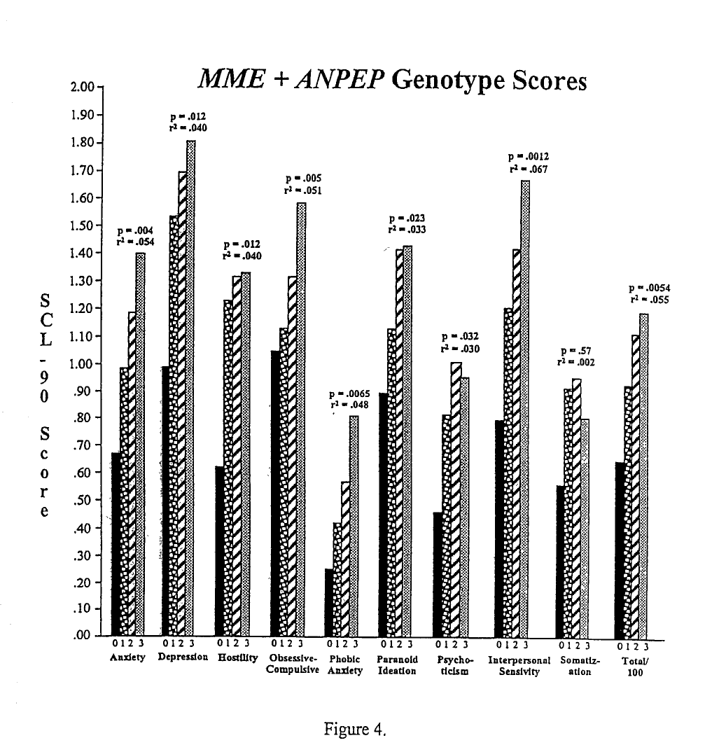

FIG. 4 shows the ANOVA results for the SCL-90 scores for the combined MME+ANPEP scores. Thus, those with a score of 0 carried the MME 4/4 and the ANPEP 11 or 12 genotype. Those with a score of 1 carried the MME 4/non4 and the ANPEP 11 or 12 genotype. Those with a score of 2 carried either the MME non4/non4 or the ANPEP 22 genotype. Finally, those with a score of 3 carried the ANPEP 22 genotype and either the 4/4 or the 4/non4 MME genotype. A SCL-90 score for those with a MME+ANPEP score of 3 that was higher than for those with a score of 2 would be indicative of an additive effect of the two genes. FIG. 4 shows a progressive increase in the scores for anxiety, depression, obsessive-compulsive, phobic anxiety, paranoid ideation, interpersonal sensitivity and the total score across the four MME+ANPEP groups. The linear ANOVA p values were significant for all of these SCL-90 scores with the lowest p values for anxiety (0.004), obsessive-compulsive (0.0065), interpersonal sensitivity (0.0012), and the total score (0.0054).

The results of the linear regression analyses are shown in FIG. 4. They showed that when the effects of the MME and ANPEP genes were combined, except for somatization, they accounted for 3.0 to 6.7 percent of the variance of the remaining scores. The highest value was for intrapersonal sensitivity (r²=0.067).

### Example 6

### NEO-FFI and Negative Affect

Since the studies of Huss et al. (1998) showed that elevated NEP levels were significantly associated with negative affect, we examined the negative affect subscore of the NEO-FFI based on the formulation of Saucier et al. (1998). This was composed of NEO-FFI items 1, 11, 16, 31 and 46. For the MME gene the results were 10.24±5.1 for the 4/4 genotype, 12.09±3.9 for the 4/non4 genotype, and 12.7±4.4 for the non4/non4 genotype, F-ratio for linear ANOVA=5.56, p=0.019. Since there was no association of the ANPEP gene with negative affect, p=0.51, the linear ANOVA for the MME+ANPEP scores was not significant.

Discussion. The present results support an important role of opioids, and the enkephalin system in particular, in psychiatric disorders, especially internalizing disorders consisting of depression, negative affect, phobic anxiety, and obsessive compulsive symptoms. Since one of the most effective ways of regulating the levels of neurotransmitters and neuropeptides is to inhibit their rate of degradation, we were especially interested in examining the potential role of allelic variants of the enzymes that degrade enkephalins, as risk factor for internalizing disorders. NEP is a carboxypeptidase and hydrolyzes enkephalins at the Gly-Phe bond (Roques et al., 1993; Sullivan et al., 1978) while APN is an aminopeptidase and degrades enkephalins at the Tyr-Gly bond (Roques et al., 1993; Sullivan et al., 1978). The results for the MME gene suggest that the 4 allele is associated with lower levels of NEP and higher CNS levels of enkephalins, while the non4/non4 genotype is putatively associated with higher NEP levels and the lower CNS levels of enkephalins. The results for the ANPEP gene suggest that the 22 genotype is associated with lower levels of APN and higher levels of CNS enkephalins while the 11 and 12 genotypes are associated with the obverse. Since each enzyme is independently capable of enkephalin degradation we hypothesized that genetic defects of both genes were more likely to result in increased CNS enkephalin levels than defects of only one gene. Thus, we hypothesized that the non4 alleles of the MME gene and the 2 alleles of the ANPEP gene would have an additive effect on internalizing phenotypes. This was tested by assigning the genotypes of each gene a score from 0 to 2, based on the results of the individual ANOVAs, and adding the scores together. When truncated to a score ranging from 0 to 3 the results (FIG. 4) for most of the internalizing scores showed a progressive linear increase in the magnitude of the SCL-90 scores across the four MME+ANPEP gene scores. Since a score of 3 could only be obtained if an individual carried the risk alleles of both genes, the finding that most of the SCL-90 scores were highest for those with a MME+ANPEP score of 3, supported the hypothesis of an interaction between the two genes.

Linear regression analysis showed that the MME+ANPEP genes accounted for up to 6.7 percent of the variance of the interpersonal sensitivity score and 3 percent or more of the variance for 9 of the 10 SCL-90 scores. Most psychiatric disorders and psychological traits are polygenically inherited (Plomin et al., 1994; Comings et al., 1996). In our experience based on examining the role of over 40 genes in a range of behavioral phenotypes, most genes account for less than 2 percent of the variance and often less than 1 percent, and even adding the effects of two genes rarely accounts for more than 2 percent of the variance. Thus, the present results showing that the additive effect of the MME+ANPEP genes accounted for 3.0 to 6.7 percent of the variance of the SCL-90 scores represents an unusually strong effect.

These results have implications for treatment. A drug that is able to pass the blood-brain barrier and inhibit both the NEP and APN enzymes would be particularly effective in the treatment of a number of psychiatric disorders including chronic depression, dysthymia, anxiety, phobia, chronic pain, general anhodenia and a range of addictive behaviors. There is an extensive literature indicating that such compounds have already been identified (Roques et al., 1993). One of the most promising is RB 101, a prodrug that passes the blood brain barrier and inhibits both NEP and APN (Noble et al., 1992; Ortega-Alvaro et al., 1998). It has been effective in the ameloriation of pain (Ortega-Alvero et al., 1998) and depression (Tejedor-Real, et al., 1998) in animals and has been reported to be devoid of the usual side-effects of opiate related drugs (Tejedor-Real, et al., 1998) in humans.

### Example 7

### Analysis of P300 Amplitude and MME Genotypes

Subjects. Twenty-five Caucasian male patients on the Addiction Treatment Unit of the Jerry L. Pettis Veterans Administration Hospital at Loma Linda, Calif. were studied. The subjects consisted of the following diagnostic categories: 6 alcohol dependence, 9 alcohol and amphetamine dependence, 2 alcohol and marijuana dependence, 1 alcohol and LSD dependence, 1 alcohol and heroin dependence, 2 amphetamine dependence, 2 heroin dependence, and 2 cocaine dependence.

Electrophysiological Methods. The auditory ERP studies were performed using the QSI-9000 computer system (Quantified Signal Imaging, Toronto, Canada). The electrode placement was through the use of an electrode cap (Electro-Cap, International, Eaton, Ohio) conforming to the international 10-20 system of electrode placement. Forehead ground with linked-ear reference electrodes were utilized in the paradigm. Eye-movement artifacts were monitored by recording the electro-oculogram (EOG) from two electrodes placed at the upper and outer canthus of the left eye. Trials with excessive eye-blink were automatically rejected and were not included in the averages. Impedance was kept well below 8 kω per electrode with overall averages per subject at about 5 kω. The auditory ERP utilized an “oddball” design with the rare tone presented randomly 20% of the time. Subjects were asked to eat a light meal two hours before testing. Subjects were asked to attend and discriminate between rare and frequent tones by a finger-raise following presentation of the rare tone.

MME Polymorphism. The MME polymorphism was analyzed as described in Example 1.

Statistical Analysis. We compared the mean P300 amplitude for the frontal (Fz), parietal (Pz) and coronal (CZ) electrodes for the different MME genotypes using ANOVA from the SPSS Statistical Packages (SPSS, Inc., Chicago, Ill.). Both standard F-ratio and p value and linear trend F-ratio and p value were examined. The correlation coefficient, r, and percent of the variance, r², were determined by univariate linear regression analysis.

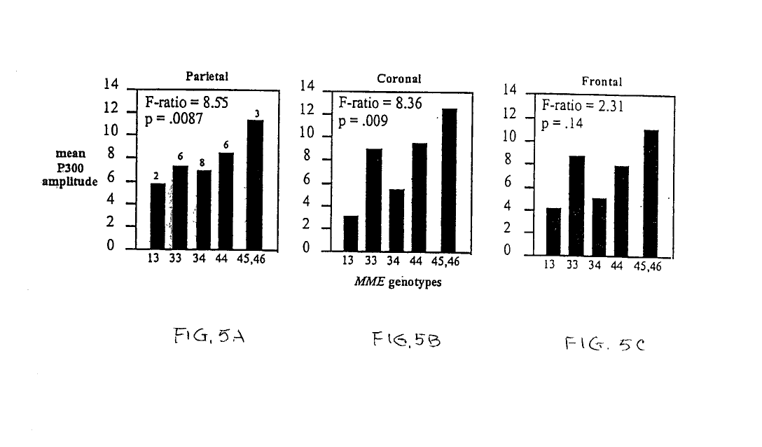

Results. There were 6 MME alleles representing 21 to 26 GT repeats. The two major alleles, 3 and 4, represented 51.3 and 37.9% of the total, respectively. Together they accounted for the 89.2% of the alleles. FIG. 5 shows the number and distribution of the MME genotypes against the P300 amplitude for the coronal, parietal and frontal leads. The genotypes were arranged in order of increasing mean size of the respective alleles. We have reported elsewhere that most of the short tandem repeat polymorphism we have studied show an association with a range of phenotypes on the basis of size (Comings, 1998). A total of 25 subjects were tested. The association of the MME genotypes with the P300 wave amplitudes of the parietal and coronal leads were significant by linear ANOVA at p<0.01, and by standard ANOVA at p<0.025. The trends were similar for the frontal leads but were not significant. To obtain an estimate of the percent of the variance attributable to the MME gene the alleles were coded as 13=1, 33=2, 34=3, 44=5 and 45 and 46=6. Linear regression analysis gave the following results: parietal r=0.50, r²=0.26, p=0.01; coronal r=0.46, r²=0.22, p=0.02; and frontal r=0.23, r²=0.08, p=0.17, suggesting the MME gene makes a substantial contribution to the amplitude of the P300 wave (8 to 25 percent of the variance). There was no association between the MME genotypes with P300 wave latency.

Discussion. While this is the first study to implicate enkephalins in general and the MME gene in particular as playing a role in the amplitude of the P300 waves, enkephalins and endorphins have frequently been implicated in alcoholism (Blum, 1985; Gianoulakis et al, 1996; Wand et al., 1998; Blum et al., 1987; Blum et al., 1981), low amplitude P300 waves are associated with familial alcoholism (Begleiter et al., 1984; Polich et al., 1994; Benegal et al., 1995; Hill et al., 1995), and the MME gene is associated with alcoholism (unpublished). Thus, the association of P300 wave amplitude with enkephalin metabolism is not surprising. Based on our other studies (Johnson et al., 1998), and those of Huss et al. (1998), we presume that the lower molecular weight alleles of the MME polymorphism are associated with increased levels of NEP and thus lower CNS enkephalin levels. The studies of the association of low P300 wave amplitude with alcoholism are usually based on studies of children of alcoholics and non-alcoholics. In a study of similar design, using a protocol to measure endogenous opioids based on cortisol response to naltrexone, Wand et al. (1998) concluded that individuals with a family history of alcoholism had diminished endogenous hypothalamic opioid activity.

We have previously found the genetic variations at the cannabinoid receptor gene (CNR1) were associated with the amplitude of the P300 wave (Johnson et al., 1997). Others have shown that the TaqI A1 allele of the dopamine D₂ receptor gene (DRD2) was associated with low amplitude of the P300 wave (Noble et al., 1994; Blum et al., 1994; Hill et al., 1998). While not all are positive (Bolos et al., 1990), numerous studies have also found the DRD2 gene to be associated with some forms of alcoholism (Hill et al., 1998; Blum et al., 1990; Noble et al., 1993; Blum et al., 1995), and there is an intimate interaction in the brain between enkephalinergic and dopaminergic neurons containing dopamine D₂ receptors (Kalivas, 1988; Lu et al., 1998; LeMoine et al., 1995).

### Example 8

### Generation of Polyclonal Antibody against APN

A segment of ANPEP coding sequence is expressed as fusion protein in E. coli. The overexpressed protein is purified by gel elution and used to immunize rabbits and mice using a procedure similar to the one described by Harlow and Lane (1988). This procedure has been shown to generate Abs against various other proteins (for example, see Kraemer et al., 1993).

Briefly, a stretch of ANPEP coding sequence is cloned as a fusion protein in plasmid PET5A (Novagen, Inc., Madison, Wis.). After induction with IPTG, the overexpression of a fusion protein with the expected molecular weight is verified by SDS/PAGE. Fusion protein is purified from the gel by electroelution. Identification of the protein as the ANPEP fusion product is verified by protein sequencing at the N-terminus. Next, the purified protein is used as immunogen in rabbits. Rabbits are immunized with 100 μg of the protein in complete Freund's adjuvant and boosted twice in 3 week intervals, first with 100 μg of immunogen in incomplete Freund's adjuvant followed by 100 μg of immunogen in PBS. Antibody containing serum is collected two weeks thereafter.

This procedure is repeated to generate antibodies against the ANPEP gene product (APN) having the disclosed polymorphism. These antibodies, in conjunction with antibodies to wild type APN, are used to detect the presence and the relative level of the polymorphic forms in various tissues and biological fluids.

### Example 9

### Generation of Monoclonal Antibodies Specific for APN

Monoclonal antibodies are generated according to the following protocol. Mice are immunized with immunogen comprising intact APN or APN peptides (wild type or polymorphic) conjugated to keyhole limpet hemocyanin using glutaraldehyde or EDC, as is well known.

The immunogen is mixed with an adjuvant. Each mouse receives four injections of 10 to 100 μg of immunogen and after the fourth injection blood samples are taken from the mice to determine if the serum contains antibody to the immunogen. Serum titer is determined by ELISA or RIA. Mice with sera indicating the presence of antibody to the immunogen are selected for hybridoma production.

Spleens are removed from immune mice and a single cell suspension is prepared (see Harlow and Lane, 1988). Cell fusions are performed essentially as described by Kohler and Milstein (1975). Briefly, P3.65.3 myeloma cells (American Type Culture Collection, Rockville, Md.) are fused with immune spleen cells using polyethylene glycol as described by Harlow and Lane (1988). Cells are plated at a density of 2×10⁵ cells/well in 96 well tissue culture plates. Individual wells are examined for growth and the supernatants of wells with growth are tested for the presence of APN specific antibodies by ELISA or RIA using wild type or polymorphic APN target protein. Cells in positive wells are expanded and subcloned to establish and confirm monoclonality.

Clones with the desired specificities are expanded and grown as ascites in mice or in a hollow fiber system to produce sufficient quantities of antibody for characterization and assay development.

### Example 10

### Sandwich Assay for APN

Monoclonal antibody is attached to a solid surface such as a plate, tube, bead or particle. Preferably, the antibody is attached to the well surface of a 96-well ELISA plate. 1100 μL sample (e.g., serum, urine, tissue cytosol) containing the APN peptide/protein (wild-type or polymorphic) is added to the solid phase antibody. The sample is incubated for 2 hrs at room temperature. Next the sample fluid is decanted, and the solid phase is washed with buffer to remove unbound material. 100 μL of a second monoclonal antibody (to a different determinant on APN peptide/protein) is added to the solid phase. This antibody is labeled with a detector molecule (e.g., ¹²⁵I, enzyme, fluorophore, or a chromophore) and the solid phase with the second antibody is incubated for two hrs at room temperature. The second antibody is decanted and the solid phase is washed with buffer to remove unbound material.

The amount of bound label, which is proportional to the amount of APN peptide/protein present in the sample, is quantified. Separate assays are performed using monoclonal antibodies which are specific for the wild-type APN as well as monoclonal antibodies specific for each of the polymorphisms identified in APN.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### LIST OF REFERENCES

Altschul, S F, et al. (1997). Nucl. Acids Res. 25:3389-3402.

Anand, R (1992). Techniques for the Analysis of Complex Genomes (Academic Press).

Ausubel, F M, et al. (1992). Current Protocols in Molecular Biology, (John Wiley & Sons, NY).

Bartel, P L, et al. (1993). “Using the 2-hybrid system to detect protein-protein interactions.” In Cellular Interactions in Development: A Practical Approach, Oxford University Press, pp. 153-179.

Begleiter, H, et al. (1984). Science 225:1493-1495.

Benegal, V, et al. (1995). Psychiat. Genet. 5:149-156.

Blum, K, et al. (1981). Toxicol. Eur. Res. 3:261-262.

Blum, K, et al. (1987). Alcohol 4:449-456.

Blum, K, et al. (1989). Alcohol 5:481-493.

Blum, K, et al. (1990). J. Am. Med. Assn. 263:2055-2059.

Blum, K, et al. (1994). Am. J. Hum. Genet. 55:A146.

Blum, K, et al. (1995). Pharmacogenetics 5:121-141.

Bolos, A M, et al. (1990). J. Am. Med. Assn. 26:3156-5160.

Borman, S (1996). Chemical & Engineering News, December 9 issue, pp.42-43.

Capecchi, M R (1989). Science 244:1288.

Cariello, N F (1988). Am. J. Human Genetics 42:726-734.

Chee, M, et al. (1996). Science 274:610-614.

Chevray, P M and Nathans, D N (1992). Proc. Natl. Acad. Sci. USA 89:5789-5793.

Comings, D E (1996). Polygenetic inheritance of psychiatric disorders. In: Handbook of Psychiatric Genetics, Blum, K. et al., eds. Boca Raton, Fla.: CRC Press, pp. 235-260.

Comings, D E (1998). Molecular Psychiatry 3:21-31.

Compton, J. (1991). Nature 350:91-92.

Conner, B J, et al. (1983). Proc. Natl. Acad. Sci. USA 80:278-282.

Costa, P T, Jr. & McCrae, R R (1992). Psychological Assessment 4:5-13.

Cotten, M, et al. (1990). Proc. Natl. Acad. Sci. USA 87:4033-4037.

DeRisi, J, et al. (1996). Nat. Genet. 14:457-460.

Deutscher, M (1990). Meth. Enzymology 182:83-89 (Academic Press, San Diego, Calif.).

Donehower, L A, et al. (1992). Nature 356:215.

Editorial (1996). Nature Genetics 14:367-370.

Elghanian, R, et al. (1997). Science 277:1078-1081.

Fahy, E, et al. (1991). PCR Methods Appl. 1:25-33.

Fields, S and Song, O-K (1989). Nature 340:245-246.

Finkelstein, J, et al. (1990). Genomics 7:167-172.

Fodor, S P A (1997). Science 277:393-395.

Fu, D-J, et al. (1998). Nat. Biotechnol. 16:381-384.

Gianoulakis, C, et al. (1996). J. Arch. Gen. Psychiatry, 53:250-257.

Glover, D (1985). DNA Cloning, I and II (Oxford Press).

Goding (1986). Monoclonal Antibodies: Principles and Practice, 2d ed. (Academic Press, NY).

Godowski, P J, et al. (1988). Science 241:812-816.

Grompe, M (1993). Nature Genetics 5:111-117.

Grompe, M, et al. (1989). Proc. Natl. Acad. Sci. USA 86:5855-5892.

Guthrie, G and Fink, G R (1991). Guide to Yeast Genetics and Molecular Biology (Academic Press).

Hacia, J G, et al. (1996). Nature Genetics 14:441-447.

Harlow, E and Lane, D (1988). Antibodies: A Laboratory Manual (Cold Spring Harbor Laboratory, Cold Spring Harbor, N.Y.).

Haouas, H. et al. (1995). Biochem. Biophys. Res. Comm. 207:933-942.

Hasty, P K, et al. (1991). Nature 350:243.

Hill, S Y, et al. (1995). Biol. Psychiatry 38:622-632.

Hill, S Y, et al. (1998). Biol. Psychiatry 43(1):40-51.

Huss, M (1998). Am. J. Med. Gen. (Neuropsych. Genet.) 81:549.

Huse, W D, et al. (1989). Science 246:1275-1281.

Innis, M A, et al. (1990). PCR Protocols: A Guide to Methods and Applications (Academic Press, San Diego, Calif.).

Jablonski, E, et al. (1986). Nucl. Acids Res. 14:6115-6128.

Johnson, J P, et al. (1998). Am. J. Med. Gen. (Neuropsych. Genet.) 81:524.

Kalivas, P W, et al. (1998). Ann. N.Y. Acad. Sci. 537:405-414.

Kanehisa, M (1984). Nucl. Acids Res. 12:203-213.

Kass, F, et al. (1983). Arch. Gen. Psychiatry 40:389-393.

Kinszler, K W, et al. (1991). Science 251:1366-1370.

Kohler, G and Milstein, C (1975). Nature 256:495-497.

Kraemer, F B, et al. (1993). J. Lipid Res. 34:663-672.

Landegren, U, et al. (1988). Science 242:229-237.

Lee, J E, et al. (1995). Science 268:836-844.

LeMoine, C, et al. (1995). J. Comp. Neurol. 355:426-481.

Lipshutz, R J, et al. (1995). BioTechniques 19:442-447.

Lockhart, D J, et al. (1996). Nature Biotechnology 14:1675-1680.

Lu, X-Y, et al. (1998). Neuroscience 82:767-780.

Maniatis, T, et al. (1982). Molecular Cloning: A Laboratory Manual (Cold Spring Harbor Laboratory, Cold Spring Harbor, N.Y.).

Martin, R, et al. (1990). BioTechniques 9:762-768.

Matthews, J A and Kricka, L J (1988). Anal. Biochem. 169:1.

Merrifield, B (1963). J. Am. Chem. Soc. 85:2149-2156.

Mifflin, T E (1989). Clinical Chem. 35:1819-1825.

Modrich, P (1991). Ann. Rev. Genet. 25:229-253.

Mombaerts, P, et al. (1992). Cell 68:869.

Newton, C R, et al. (1989). Nucl. Acids Res. 17:2503-2516.

Nguyen, Q, et al. (1992). BioTechniques 13:116-123.

Noble, F, et al. (1992). J. Pharmacol. Exp. Ther. 261:181-190.

Noble, E P, et al. (1993). Behav. Genet. 23:119-129.

Noble, E P, et al. (1994). Am. J. Hum. Genet. 54:658-668.

Novack, D F, et al. (1986). Proc. Natl. Acad. Sci. USA 83:586-590.

Orita, M, et al. (1989). Proc. Natl. Acad. Sci. USA 86:2766-2770.

Ortega-Alvaro, A, et al. (1998). Eur. J. Pharmacology 356:139-148.

Philpott, K L, et al. (1992). Science 256:1448.

Plomin, R, et al. (1994). Science 264:1733-1739.

Polich, J, et al. (1994). Psychological Bulletin 115:55-73.

Remington's Pharmaceutical Sciences, 18th Ed. (1990, Mack Publishing Co., Easton, Pa.).

Rigby, P W J, et al. (1977). J. Mol. Biol. 113:237-251.

Roques, B P (1993). Pharmacol. Rev. 45:37-146.

Ruano, G and Kidd, K K (1989). Nucl. Acids Res. 17:8392.

Sambrook, J, et al. (1989). Molecular Cloning: A Laboratory Manual, 2nd Ed. (Cold Spring Harbor Laboratory, Cold Spring Harbor, N.Y.).

Saucier, G. (1998). J. Pers. Assess. 70:263-270.

Scharf, S J, et al. (1986). Science 233:1076-1078.

Scopes, R (1982). Protein Purification: Principles and Practice, (Springer-Verlag, NY).

Sheffield, V C, et al. (1989). Proc. Natl. Acad. Sci. USA 86:232-236.

Shenk, T E, et al. (1975). Proc. Natl. Acad. Sci. USA 72:989-993.

Shinkai, Y, et al. (1992). Cell 68:855.

Shoemaker, D D, et al. (1996). Nature Genetics 14:450-456.

Snouwaert, J N, et al. (1992). Science 257:1083.

Spargo, C A, et al. (1996). Mol. Cell. Probes 10:247-256.

Steer, R A, et al. (1994). J. Pers. Assess. 62:525-536.

Sullivan, S, et al. (1978). Commun. Psychopharmacol. 2:525-531.

Tejedor-Real P. et al. (1998). Eur. J. Pharmacology 354:1-7.

Valancius, V and Smithies O (1991). Mol. Cell Biol. 11:1402.

Walker, G T, et al., (1992). Nucl. Acids Res. 20:1691-1696.

Wand, G S, et al. (1998). Arch. Gen. Psychiatry 55:1114-1119.

Wartell, R M, et al. (1990). Nucl. Acids Res. 18:2699-2705.

Watt, V M & Willard, H F (1990). Hum. Genet. 85:651-654.

Wetmur, J G and Davidson, N (1968). J. Mol. Biol. 31:349-370.

White, M B, et al. (1992). Genomics 12:301-306.

White, R and Lalouel, J M (1988). Annu. Rev. Genet. 22:259-279.

Wu, D Y and Wallace, R B (1989). Genomics 4:560-569.

Patents and Patent Applications:

European Patent Application Publication No. 0332435.

EPO Publication No. 225,807.

EP 425,731A.

WO 90/07936.

WO 92/19195.

WO 94/25503.

WO 95/01203.

WO 95/05452.

WO 96/02286.

WO 96/02646.

WO 96/11698.

WO 96/40871.

WO 96/40959.

WO 97/12635.

U.S. Pat. No. 3,817,837.

U.S. Pat. No. 3,850,752.

U.S. Pat. No. 3,939,350.

U.S. Pat. No. 3,996,345.

U.S. Pat. No. 4,275,149.

U.S. Pat. No. 4,277,437.

U.S. Pat. No. 4,366,241.

U.S. Pat. No. 4,376,110.

U.S. Pat. No. 4,486,530.

U.S. Pat. No. 4,683,195.

U.S. Pat. No. 4,683,202.

U.S. Pat. No. 4,816,567.

U.S. Pat. No. 4,868,105.

U.S. Pat. No. 5,270,184.

U.S. Pat. No. 5,409,818.

U.S. Pat. No. 5,455,166.

U.S. Pat. No. 5,550,050.

U.S. Pat. No. 5,800,998.

U.S. Pat. No. 5,837,492.

U.S. Pat. No. 5,891,628.

What is claimed is:

1. A method for diagnosing a polymorphism which causes an internalizing disorder comprising hybridizing a nucleic acid probe which hybridizes specifically to a nucleic acid selected from the group of (a) a nucleic acid comprising a nucleotide sequence coding for human MME containing a polymorphism described herein or its complement and (b) a nucleic acid comprising a nucleotide sequence coding for human APN containing a polymorphism described herein or its complement to a patient's sample of DNA or RNA under stringent conditions which allow hybridization of said probe to nucleic acid comprising said polymorphism but prevent hybridization of said probe to a wild-type nucleic acid, wherein the presence of a hybridization signal indicates the presence of said polymorphism.

2. The method according to claim 1 wherein the patient's DNA or RNA has been amplified and said amplified DNA or RNA is hybridized.

3. A method according to claim 2 wherein hybridization is performed in situ.

4. A method for diagnosing the presence of a polymorphism in human MME or ANPEP which causes an internalizing disorder wherein said method is performed by means which identify the presence of a polymorphism selected from the group described herein.

5. The method of claim 4 wherein said means comprises using a single-stranded conformation polymorphism technique to assay for said polymorphism.

6. The method of claim 4 wherein said means comprises sequencing human MME or ANPEP.

7. The method of claim 4 wherein said means comprises performing an RNase assay.

8. An antibody which binds to a polymorphic APN polypeptide but not to wild-type APN polypeptide, wherein said polymorphic APN has an altered sequence as disclosed herein.

9. A method for diagnosing an internalizing disorder comprising an assay for the presence of polymorphic APN polypeptide in a patient by reacting a patient's sample with an antibody of claim 8 wherein the presence of a positive reaction is indicative of an internalizing disorder.

10. The method of claim 9 wherein said antibody is a monoclonal antibody.

11. The method of claim 9 wherein said assay comprises immunoblotting or an immunocytochemical technique.

12. An isolated polypeptide an amino acid sequence of APN with a polymorphism described herein.

13. A host comprising a nucleic acid selected from the group of (a) a nucleic acid comprising a nucleotide sequence coding for human MME containing a polymorphism described herein or its complement and (b) a nucleic acid comprising a nucleotide sequence coding for human APN containing a polymorphism described herein.

14. The host of claim 13 which is a transformed or transfected cell.

15. The host of claim 13 which is a nonhuman, transgenic animal.

16. A method of correlating a placebo response to a polymorphism described herein which comprises i.providing a placebo to a cell or animal having said polymorphism and detecting whether a placebo response is present, whereby the presence or absence of a placebo response is correlated to the said polymorphism.

17. A method of correlating a polymorphism described herein with a drug that inhibits the activity of NEP which comprises providing said drug to a cell or animal having said polymorphism and detecting inhibition of NEP, whereby inhibition of NEP is correlated to said polymorphism and said drug is useful for treating a disorder associated with said polymorphism.

18. A method of correlating a polymorphism described herein with a drug that inhibits the activity of APN which comprises providing said drug to a cell or animal having said polymorphism and detecting inhibition of APN, whereby inhibition of APN is correlated to said polymorphism and said drug is useful for treating a disorder associated with said polymorphism.

19. A method to screen for drugs which are useful in treating a person with an internalizing disorder from a polymorphism in MME and/or ANPEP as described herein, wherein said method comprises providing said drug to a cell or animal having said polymorphism and detecting inhibition of NEP and/or APN, whereby inhibition of NEP and/or APN is indicative that said drug is useful for treating a disorder associated with said polymorphism.

20. A method to screen for drugs which are useful in treating or preventing an internalizing disorder, said method comprising:
(a) preparing a transgenic animal comprising an MME gene and/or ANPEP gene having a polymorphism described herein;
(b) measuring the level of enkephalins in the CNS of the animals of step (a);
(c) administering a drug to the transgenic animal of step (a);
(d) measuring the level of enkephalins in the CNS of the animals of step (c); and
(e) comparing the level of enkephalins in the CNS of steps (b) and (d), wherein a drug which increases the levels of enkephalins in the CNS is useful in treating or preventing an internalizing disorder.
